(19) United States
(12) Patent Application Publication
(10) Pub. No.: US 2014/0186290 A1
(43) Pub. Date: Jul. 3, 2014

(54) NANOPARTICLE BASED THERAPY FOR DISPERSING MUCIN

(71) Applicant: The Regents of the University of California, Oakland, CA (US)

(72) Inventors: Wei-Chun Chin, Merced, CA (US); Eric Yi-Tong Chen, Merced, CA (US); Yung-Chen Wang, Merced, CA (US)

(73) Assignee: The Regents of the University of California, Oakland, CA (US)

(21) Appl. No.: 14/201,357

(22) Filed: Mar. 7, 2014

Related U.S. Application Data
(62) Division of application No. 12/958,738, filed on Dec. 2, 2010.
(60) Provisional application No. 61/266,295, filed on Dec. 3, 2009.

(51) Int. Cl. A61K 31/745

(52) U.S. Cl. CPC A61K 31/745
USPC 424/78.31

(57) ABSTRACT

There are provided compositions and methods to disperse mucin and/or actin using nanoparticles wherein the average diameter of the nanoparticles is less than about 1000 nm.

### CROSS REFERENCE TO RELATED APPLICATION

This application is a divisional of U.S. patent application Ser. No. 12/958,738, filed Dec. 2, 2010, which claims the benefit under 35 U.S.C. §119(e) of U.S. Provisional Ser. No. 61/266,295, filed Dec. 3, 2009. The contents of both applications are hereby incorporated by reference in their entirety for all purposes.

### FIELD OF THE INVENTION

This disclosure relates to compositions and methods for dispersing mucin and/or actin in a subject suffering from an aggregation of mucus in different parts of the body, such as, but not limited to, eyes, ears, pancreatic ducts, gallbladder, prostate, respiratory, gastrointestinal and male and female reproductive tracts.

### BACKGROUND

Mucins are a family of glycoproteins secreted from epithelial cells at body surfaces, including the eyes, pancreatic ducts, gallbladder, prostate, respiratory, gastrointestinal and female reproductive tracts. Mucins provide the viscoelastic properties to mucus. In the airways, mucin interacts with cilia to trap and clear pathogens and irritants. Bacterial infection of the airway epithelium is often accompanied by mucin overproduction. In addition, airway diseases such as chronic bronchitis, cystic fibrosis and asthma are characterized by mucus hypersecretion. Hypersecretion can overwhelm the ability of the cilia to function properly. Mucus overproduction resulting from the bacterial infection damages lung function directly by plugging airways and indirectly by shielding the bacteria from endogenous and exogenous antibacterial agents. This creates a wound that does not heal and causes chronic influx of inflammatory cells whose proteases degrade gas exchange tissue. Respiratory function declines relentlessly until death results.

Current treatments are not effective for complete eradication or prevention of these bacterial infections nor to ameliorate the overproduction of mucus. In addition, anti-microbial therapy using antibiotic therapeutic protocols have complications. Patients with cystic fibrosis (CF) dispose of anti-microbial agents more rapidly than do non-CF individuals, which results in the use of higher doses of antibiotics than normally recommended. Therapeutic levels of anti-microbial agents in sputum are difficult to achieve because of poor penetration and inactivation. Finally, allergy to certain antibiotics (such as beta-lactam) precludes antibiotic therapy with some patients. Thus, it is important to find alternate therapies to improve lung function and prolong life. The ability to control the aggregation of mucin may provide an alternative route to prevent or alleviate airway plugging.

In addition to its role in exacerbating pulmonary infections in cystic fibrosis patients, mucin overproduction is also a debilitating feature of chronic bronchitis, bronchial pneumonia and chronic asthma. Smoking is an important risk factor for chronic bronchitis. Individuals dying in status asthmaticus are observed to have mucus-obstructed airways. In addition, mucus is also implicated in various eye and ear infections and infections related to rectal and vaginal tract.

Consequently, there is a need to provide therapies for treating or reducing mucus accumulation in individuals.

### SUMMARY OF THE INVENTION

Mucus clearance is the first line of pulmonary defense against inhaled irritants, microorganisms, and allergens. Disclosed herein are compositions and methods using nanoparticles for a dispersion of mucin in subjects suffering from an aggregation of mucus in different parts of the body, such as, but not limited to, eyes, ears, pancreatic ducts, gallbladder, prostate, respiratory, gastrointestinal and male and female reproductive tracts. Increased mucin production can also occur in many adenocarcinomas, including cancer of the pancreas, lung, breast, ovary, colon, etc. Mucins may also be overexpressed in lung diseases such as asthma, bronchitis, COPD (chronic obstructive pulmonary disease) or cystic fibrosis (CF). The compositions and methods of nanoparticles provided herein, avoid the need for drugs, chemicals or enzymes which are conventionally used to disperse the mucin.

The disclosure provides compositions and methods for the dispersion of mucin in a subject by administration of a composition of nanoparticles having an overall negative charge. In one aspect, the nanoparticles are negatively charged. In some embodiments, the nanoparticles are negatively charged polystyrene nanoparticles. Without being bound by theory, it is proposed that the mucin in a subject is aggregated or gelated with a cross-linking network of positively charged ions, such as, but not limited to, potassium, calcium or magnesium ions. For example, Ca²⁺ ions or Mg²⁺ ions cross link with the negatively charged polyglcosylated mucin by electrostatic attraction resulting in the aggregation or gelation of mucin. The administration of the composition causes the chelation of the positively charged ions on mucin with the negatively charged nanoparticles thereby causing the dispersion of the aggregate of mucin. Without being bound by theory, when the nanoparticles have an overall negative charge, Applicants submit that due to polyglycosylated nature of mucin, the negative charged mucin electrostatically repels the negatively charged nanoparticle which further results in the dispersion of the mucin. The chelation of the negatively charged nanoparticles with the positively charged ions reduces the availability of positively charged ions for cross linking with mucin and prevents mucin gel formation.

The compositions and methods of the disclosure have various advantages including, but are not limited to, drug free composition, economical manufacturing, reduced side effects, ease of delivery, and ease of modification based on the subject and the body surface.

Applicants also show that nanoparticles having an overall positive charge also disperse actin and in a relative lower concentration than negatively charged nanoparticles. Thus, in another aspect, the disclosure provides compositions and methods for the dispersion of actin in the mucus of a subject by administration of a composition of nanoparticles. In one aspect the plurality have an overall negative charge. In another aspect, the plurality of nanoparticles have an overall positive charge. The gel-forming mucins are the principal polymeric components of airway mucus but in cystic fibrosis (CF), the necrotic death of inflammatory and epithelial cells releases a network of copolymerized extracellular DNA and filamentous (F-) actin-producing secretions that are similar to pus and difficult to clear by cilia or airflow. To improve drug delivery in CF it is vital to reduce or remove this barrier.

In one aspect, there is provided a composition comprising, or alternatively consisting essentially of, or yet further consisting of, a plurality of nanoparticle, wherein the average diameter of the nanoparticles is less than about 500 nm. In one aspect the plurality of nanoparticles have an overall negative charge. In another aspect, the plurality of nanoparticles have an overall positive charge. In some embodiments, the nanoparticles of the plurality comprises polystyrene nanoparticles that are negatively charged or positively charged.

In another aspect, there is provided a method to disperse mucin in a subject comprising, or alternatively consisting essentially of, or yet further consisting of, administering to the subject an effective amount of the composition comprising, or alternatively consisting essentially of, or yet further consisting of, a plurality of nanoparticles wherein the average diameter of the nanoparticles is less than about 500 nm, thereby dispersing the mucin. In one aspect the plurality have an overall negative charge. In another aspect, the plurality of nanoparticles have an overall positive charge.

In another aspect, there is provided a method to disperse actin in a mucus of a subject comprising, or alternatively consisting essentially of, or yet further consisting of, administering to the subject an effective amount of the composition comprising, or alternatively consisting essentially of, or yet further consisting of, a plurality of nanoparticles, wherein the average diameter of the nanoparticles is less than about 500 nm, thereby dispersing the actin in the mucus of the subject. In one aspect the plurality have an overall negative charge. In another aspect, the plurality of nanoparticles have an overall positive charge.

In another aspect, there is provided a method to treat a disease or condition caused by an aggregation of mucin in a subject, comprising, or alternatively consisting essentially of, or yet further consisting of, administering to the subject a therapeutically effective amount of a composition comprising, or alternatively consisting essentially of, or yet further consisting of, a plurality of nanoparticles wherein the average diameter of the nanoparticles is less than about 500 nm, thereby treating the disease in the subject. In one aspect the plurality have an overall negative charge. In another aspect, the plurality of nanoparticles have an overall positive charge.

In another aspect, there is provided a method to treat a disease caused by an aggregation of actin in a mucus of a subject, comprising, or alternatively consisting essentially of, or yet further consisting of, administering to the subject a therapeutically effective amount of a composition comprising, or alternatively consisting essentially of, or yet further consisting of, a plurality of nanoparticles wherein the average diameter of the nanoparticles is less than about 500 nm, thereby treating the disease in the subject. In one aspect the plurality have an overall negative charge. In another aspect, the plurality of nanoparticles have an overall positive charge.

The compositions can be topically applied in the methods of the disclosure or alternatively, administered by conventional inhalation therapy. By varying the size of the nanoparticle, differential effects in reaching different depth of penetration within the affected tissue or organ can be achieved. For example, by varying the particle size, various depths of the bronchial tree are penetrated.

In another aspect, there is provided an in vitro method to disperse mucin, comprising, or alternatively consisting essentially of, or yet further consisting of, administering to the mucin a composition comprising a plurality of nanoparticles wherein the average diameter of the nanoparticles is less than about 500 nm. In one aspect the plurality have an overall negative charge. In another aspect, the plurality of nanoparticles have an overall positive charge.

In another aspect, there is provided an in vitro method to disperse actin in a mucus, comprising, or alternatively consisting essentially of, or yet further consisting of, administering to the actin in the mucus a composition comprising a plurality of nanoparticles wherein the average diameter of the nanoparticles is less than about 500 nm. In one aspect the plurality have an overall negative charge. In another aspect, the plurality of nanoparticles have an overall positive charge.

In another aspect, there is provided a pharmaceutical dosage form to disperse a mucin, comprising, or alternatively consisting essentially of, or yet further consisting of, an effective amount of a plurality of nanoparticles having an overall negative charge wherein the average diameter of the nanoparticles is less than about 500 nm. In one aspect the plurality have an overall negative charge. In another aspect, the plurality of nanoparticles have an overall positive charge.

In another aspect, there is provided a pharmaceutical dosage form to disperse a mucin, comprising, or alternatively consisting essentially of, or yet further consisting of, an effective amount of a plurality of nanoparticles wherein the average diameter of the nanoparticles is less than about 500 nm. In one aspect the plurality have an overall negative charge. In another aspect, the plurality of nanoparticles have an overall positive charge.

In another aspect, there is provided a pharmaceutical dosage form to disperse an actin in a mucus, comprising, or alternatively consisting essentially of, or yet further consisting of, an effective amount of a plurality of nanoparticles wherein the average diameter of the nanoparticles is less than about 500 nm.

In another aspect, there is provided a kit comprising, or alternatively consisting essentially of, or yet further consisting of, a composition comprising a plurality of nanoparticles having an overall negative charge wherein the average diameter of the nanoparticles is less than about 500 nm; and instructions for use.

In another aspect, there is provided a device for a delivery of a composition for dispersing mucin or actin, wherein the device comprises, or alternatively consists essentially of, or yet further consists of, an effective amount of a composition comprising a plurality of nanoparticles wherein the average diameter of the nanoparticles is less than about 500 nm. In one aspect the plurality have an overall negative charge. In another aspect, the plurality of nanoparticles have an overall positive charge.

### BRIEF DESCRIPTION OF THE DRAWINGS

FIG. 1 illustrates F-actin network (F-actin+MgCl₂), before the addition of any nanoparticles.

FIG. 2 illustrates the effect of 10 g/L hydrophobic polystyrene nanoparticles on F-actin network.

FIG. 3 illustrates the effect of 100 mg/L hydrophobic polystyrene nanoparticles on F-actin network.

FIG. 4 illustrates the effect of 10 g/L positively charged amine surface modified polystyrene nanoparticles on F-actin network.

FIG. 5 illustrates the effect of 100 mg/L positively charged amine surface modified polystyrene nanoparticles on F-actin network.

FIG. 6 illustrates the effect of 10 g/L negatively charged carboxyl surface modified polystyrene nanoparticles on F-actin network.

FIG. 7 illustrates the effect of 100 mg/L negatively charged carboxyl surface modified polystyrene nanoparticles on F-actin network.

### DETAILED DESCRIPTION OF THE INVENTION

### Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); Current Protocols in Immunology (J. E. Coligan, et. al. eds., (1997)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a nanoparticle” includes a plurality of nanoparticles, including mixtures thereof.

An “administration” refers to the delivery of a medication, such as the composition of nanoparticles of the disclosure to an appropriate location of the subject or in vitro, where a desired effect is achieved. Non-limiting examples include topical, rectal, vaginal, inhalation, buccal, ocular, oral, intracutaneous injection, direct application to target area proximal areas on the skin, or applied on a patch. Various physical and/or mechanical technologies are available to permit the sustained or immediate topical or transdermal administration of nanoparticles.

“Comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

An “effective amount” or a “therapeutically effective amount” is an amount sufficient to effect beneficial or desired results. An effective amount can be administered in one or more administrations, applications or dosages.

The term “nanoparticle” as used herein denotes a structure which is biocompatible with and sufficiently resistant to chemical and/or physical destruction by the environment of use such that a sufficient amount of the nanoparticles remain substantially intact after delivery to the site of application or treatment or incubated with an in vitro sample so as to be able to reach mucin or actin or reach the nucleus of a cell or some other cellular structure. The nanoparticles may undergo biodegradation upon dispersal of mucin or actin or upon entry of a cell's nucleus.

A “composition” is intended to include the combination of an active agent with a carrier, inert or active such as saline or water, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

“Topical administration” refers to delivery of a medication by application to the mucosal membrane or skin. Non-limiting examples of topical administration include any methods described under the definition of “administration” pertaining to delivery of a medication to appropriate area.

For inhalation use, the pharmaceutically acceptable carrier is suitable for manufacture of inhalers, aerosols, vaporizers, nebulizers, or lavage delivery. For topical use, the pharmaceutically acceptable carrier is suitable for manufacture of, creams, ointments, jellies, gels, solutions, suspensions, etc. Such carriers are conventional in the art. These formulations may optionally comprise additional pharmaceutically acceptable ingredients such as diluents, stabilizers, and/or adjuvants.

“Mucin” or “mucins,” as used herein, refers to the glycol-peptides of mucus secreted from epithelial cells that form mucosal barrier to protect various tissues, such as the eyes, pancreas, intestine, exocrine glands, hepatobiliary, respiratory and reproductive tracts. There are approximately 20 different types of mucins known in the art, e.g. MUC 1, MUC 2, MUC 5AC and MUC 5B . . . etc. Typically, mucins form extremely large oligomers through linkage of glycoprotein monomers using disulfide bonds. Usually, such glycoproteins are large >100,000 daltons and typically consist of approximately 75% carbohydrate and 25% protein. Altered mucins, which contain abnormal concentration of sulfate, sialic acid or fucose, also occur in pathological conditions, such as inflammatory diseases.

“Mucus” as used herein, refers to the mixtures of different types of mucins, actins, DNA or other glycol-proteins that form the hydrated layer on the surface of various tissues, such as the eyes, pancreas, intestine, exocrine glands, hepatobiliary, respiratory and reproductive tracts.

A “subject” of diagnosis or treatment is a cell or a mammal, including a human. Non-human animals subject to diagnosis or treatment include, for example conventional animal models such as murine, such as rats, mice, canine, such as dogs, leopards, such as rabbits, livestock, sport animals, frogs, and pets.

As used herein, the terms “treating,” “treatment” and the like are used herein to mean obtaining a desired pharmacologic and/or physiologic effect. The effect can be prophylactic in terms of completely or partially preventing an infection or disease or disorder or sign or symptom thereof, and/or can be therapeutic in terms of a partial or complete cure for a disorder and/or adverse effect attributable to the disorder. Examples of “treatment” include but are not limited to: preventing a disease from occurring in a subject that may be predisposed or at risk of an infection or a disease, but has not yet been diagnosed as having it; inhibiting a disease, i.e., arresting its development; and/or relieving or ameliorating the symptoms of disease or reducing the likelihood of recurrence of the disease, e.g., respiratory diseases. As is understood by those skilled in the art, “treatment” can include systemic amelioration of the symptoms associated with the pathology and/or a delay in onset of symptoms.

### Compositions

In one aspect, there is provided a composition comprising, or alternatively consisting essentially of, or yet further consisting of, a plurality of nanoparticles wherein the average diameter of the nanoparticles is less than about 500 nm. In one aspect the plurality have an overall negative charge. In another aspect, the plurality of nanoparticles have an overall positive charge. The composition of the disclosure can be used to disperse mucin in a subject. In some embodiments, the nanoparticle is negatively charged. In some embodiments, the nanoparticle is a polystyrene nanoparticle that is negatively charged.

Nanoparticles can be solid colloidal particles ranging in size from 1 to 1000 nm. Nanoparticles of the present disclosure can have any diameter less than or equal to 5 nm, or alternatively less than about 10 nm, or alternatively less than about 15 nm, or alternatively less than about 20 nm, or alternatively less than about 25 nm, or alternatively less than about 30 nm, or alternatively less than about 50 nm, or alternatively less than about 100 nm, or alternatively less than about 150 nm, or alternatively less than about 200 nm, or alternatively less than about 300 nm, or alternatively less than about 400 nm, or alternatively less than about 500 nm. In some embodiments, the nanoparticle has a diameter between about 100 nm to about 500 nm; or alternatively between about 50 nm to about 200 nm; or alternatively between about 150 nm to about 300 nm; or alternatively between about 200 nm to 500 nm; or alternatively between about 100 nm to about 150 nm; alternatively between about 1 nm to about 50 nm; alternatively between about 50 nm to about 100 nm; alternatively between about 150 nm to about 200 nm; alternatively between about 200 nm to about 300 nm; alternatively between about 300 nm to about 400 nm; or alternatively between about 400 nm to about 500 nm. Drugs, bioactive or other relevant materials can be incubated with the nanoparticles, and thereby be adsorbed or attached to the nanoparticle.

There are a number of advantages of the present disclosure. One advantage of the present technology is the ability to use nanoparticles that comprise a material that is biologically inert and can be physiologically tolerated without significant adverse effects by biological systems. Further, a nanoparticle can be comprised of a biodegradable material. There are no limits on the physical parameters of a nanoparticle component of the present disclosure, although the design of a delivery vehicle should take into account the biocompatibility of the nanoparticle vehicle and the effect on the overall charge. The physical parameters of a nanoparticle vehicle can be optimized, with the desired effect governing the choice of size and shape. For example, the nanoparticle sizes for transport to a cell's nucleus can be on the order of 5 nm where larger particles would be desired for a given application. Additionally, particles smaller than about 25 nm in diameter can be used in nuclear targeting to facilitate entry into the nucleus via a nuclear pore.

The nanoparticle can comprise a variety of materials including, but not limited to, polymers such as polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also suitable for use as a nanoparticle scaffold. The disclosure encompasses any nanoparticle that is negatively charged. The nanoparticles may themselves have a negative charge or alternatively a positive charge on them or may be modified to attach a negative charge or positive charge to the scaffold, such as, but not limited to, aldehyde, amine, carboxyl, sulfate, or hydroxyl groups. Factors such as nanoparticle surface charge and hydrophilic/hydrophobic balance of these polymeric materials can be achieved by synthetic modification of the polymers. Such synthetic modification is well within the skill of the skilled artisan, examples of which are described below. Various methods for producing the negatively charged nanoparticles are described in U.S. Pat. No. 7,390,384; and Kim et al. (2009) Polymer Bulletin 62:23-32, which are incorporated herein by reference in their entirety.

Examples of negatively charged nanoparticles include, but are not limited to, polymer blends of poly(lactide-co-glycolide) (PLGA) and poly(styrene-co-4-styrene-sulfonate) (PSS) and polystyrene nanoparticles modified with amine, carboxylate, sulfate, hydroxyl, or aldehyde-sulfate. In some embodiments, the surface charge on the nanoparticle is from about 0.1 μeq/g to about 2000 μeq/g; about 1 μeq/g to about 1000 μeq/g; about 1 μeq/g to about 900 μeq/g; about 1 μeq/g to about 800 μeq/g; about 1 μeq/g to about 700 μeq/g; about 1 μeq/g to about 600 μeq/g; about 1 μeq/g to about 500 μeq/g; about 1 μeq/g to about 400 μeq/g; about 1 μeq/g to about 300 μeq/g; about 1 μeq/g to about 200 μeq/g; about 1 μeq/g to about 100 μeq/g; about 0.1 μeq/g to about 1000 μeq/g; or about 0.1 μeq/g to about 100 μeq/g. In some embodiments, for the carboxyl surface modified polymer, the surface charge ranges from about 1 μeq/g to about 1000 μeq/g. In some embodiments, for the amine surface modified polymer, the surface charge ranges from about 1 μeq/g to about 100 μeq/g.

The surface charge of the nanoparticle and the zeta potential can be determined using a Malvern Zetamaster. Zeta potential is an abbreviation for electrokinetic potential in colloidal systems. Zeta potential is the potential difference between the dispersion medium and the stationary layer of fluid attached to the dispersed particle. The value of zeta potential can be related to the stability of colloidal dispersions (e.g. an expectorant syrup). The zeta potential indicates the degree of repulsion between adjacent, similarly charged particles in a dispersion. For molecules and particles that are small enough, a high zeta potential will confer stability, i.e. the solution or dispersion will resist aggregation. When the potential is low, attraction exceeds repulsion and the dispersion will break and flocculate. So, colloids with high zeta potential (negative or positive) are electrically stabilized while colloids with low zeta potentials tend to coagulate or flocculate.

Nanoparticles comprising the above materials and having diameters less than 1,000 nanometers are available commercially or they can be produced from progressive nucleation in solution (e.g., by colloid reaction), or by various physical and chemical vapor deposition processes, such as sputter deposition.

Besides sputter deposition, plasma-assisted chemical vapor deposition (PACVD) is another technique that can be used to prepare suitable nanoparticles. PACVD functions in relatively high atmospheric pressures (on the order of one torr and greater) and is useful for generating particles having diameters of about 1000 nanometers and smaller. The PACVD system typically includes a horizontally mounted quartz tube with associated pumping and gas feed systems. A susceptor is located at the center of the quartz tube and heated using a 60 KHz radio frequency source. The synthesized particles are collected on the walls of the quartz tube. Nitrogen gas is commonly used as the carrier. A constant pressure in the reaction chamber of 10 torr is generally maintained to provide deposition and formation of the ultrafine nanoparticles. PACVD can be used to prepare a variety of suitable biodegradable nanoparticles.

### Diagnostic and Therapeutic Utilities

In one aspect, there is provided a method to disperse mucin, comprising, or alternatively consisting essentially of, or yet further consisting of, contacting the mucin with an effective amount of the composition disclosed herein, thereby dispersing the mucin. In another aspect, there is provided a method to disperse an aggregation of mucin in a subject, comprising, or alternatively consisting essentially of, or yet further consisting of, by contacting the aggregation with an effective amount of a composition disclosed herein, thereby dispersing the aggregation. These methods can be practiced in vitro or in vivo. When practiced in vitro, they provide a suitable cell free system to test or screen for agents or formulations that may augment the inventive compositions as provided herein. To practice the screen, the mucin is prepared in two separate containers and one is contacted with the composition of this disclosure and the second is contacted with the test agent or composition and the dispersion is compared to the container containing the inventive composition. A third negative control containing the mucin and no agent or formulation may also be included. When practiced in vivo, the methods provide a therapeutic utility as described in more detail below.

In one aspect, there is provided a method to disperse mucin in a subject, comprising, or alternatively consisting essentially of, or yet further consisting of, administering to the subject an effective amount of the composition disclosed herein, thereby dispersing the mucin in the subject. In another aspect, there is provided a method to treat a disease caused by an aggregation of mucin in a subject, comprising, or alternatively consisting essentially of, or yet further consisting of, administering to the subject a therapeutically effective amount of a composition disclosed herein, thereby treating the disease in the subject. Subjects that can be treated by these methods include animals such as porcine and mice as well as human patients.

The present methods can be practiced on a variety of subjects in which there is an undesirable accumulation of mucous as a result of a medical condition. The disclosure is not to be so limited to any particular disease so long as the amount and viscosity of the mucous has risen to a degree that there are undesirable symptoms in the patient. Exemplary diseases where mucous accumulates and is producing undesirable symptoms includes, but is not limited to, common cold, inflammation of the trachea or lungs, asthma, cystic fibrosis, chronic bronchitis, pneumonia, chronic obstructive pulmonary disease, inflammation of the mucus membrane of the middle ear, inflammation of eyes, clogging of rectum, or clogging of vaginal tract, and the like.

The respiratory airways comprise a large and complex collection of organs, that encompasses all of the tissues having surfaces exposed to the passage of air during normal breathing through either the nose or mouth. Thus, the respiratory airways include air-exposed surfaces of the nasal passage, larynx, mouth, trachea, lung bronchi, lung bronchioles, lung alveolar ducts, lung alveolar sacs and lung alveoli, although the lung and associated organs are the primary target for accumulation of viscous mucous. Thus, the composition of the disclosure containing the nanoparticle can be administered to any or all of the affected tissues of the respiratory airways, although the typical and primarily affected airway by accumulation of viscous mucous is the lung, and the associated ducts, sacs and alveoli.

In healthy individuals whose lungs are uninfected, lung secretions are complex non-homogeneous materials that form a viscous hydrophilic whitish gel. Mucous glycoproteins in the uninfected lung secretions contribute to the viscosity. Cystic fibrosis patients who do not have concurrent bacterial or viral infections also exhibit increased mucous viscosity. The accumulation and persistence of high viscosity secretions contribute to respiratory distress and progressive lung destruction. Specifically, in diseases such as cystic fibrosis, airway secretions are a primary factor in respiratory dysfunction and ultimately contribute to the death of individuals with the disease. The secretions have been characterized as thick and highly viscous. As such, they are difficult to expectorate and contribute to reduced lung volumes and expiratory flow rates.

The accumulation of viscous secretions in persons with cystic fibrosis is not confined to the respiratory tract, but extends to the intestine, pancreas, biliary tract, salivary glands and genitourinary tract. This multi-system disorder is an autosomal recessive genetic disease due to mutation of the cystic fibrosis transmembrane conductance regulator (CFTR) on the long arm of chromosome 7. The CFTR is predominantly expressed in epithelial cells but mRNA can also be detected at much lower levels in leukocytes, skeletal muscle, as well as fetal liver, kidney, heart, and brain. In addition, cystic fibrosis patients, as well as persons with chronic bronchitis or pneumonia, are further characterized as having chronic infections of pseudomonas aeruginosa where despite antibiotic therapy, efficacy of treatment with aminoglycoside antibiotics is reduced.

Mucin over-production in cystic fibrosis is also present in the pancreatic ducts that deliver digestive enzymes to the GI tract resulting in malabsorption syndrome, steatorrhea and diarrhea.

Accordingly, in one aspect, there is provided a method to treat cystic fibrosis in a subject by dispersing an aggregation of mucin in the subject, comprising, or alternatively consisting essentially of, or yet further consisting of, administering to the subject a therapeutically effective amount of a composition disclosed herein, thereby treating the cystic fibrosis in the subject. Applicants also provide a method for treating cystic fibrosis by dispersing mucin in the pancreas.

Mucus in CF is more viscous than normal airway secretions, due to the high content of DNA and actin. DNA and actin are products of inflammatory cell necrosis which together with mucins form an interconnected tangled network that is held together by electrostatic, hydrogen and van der Waals forces (see Shur et al. (2009) Poster presented in Drug Delivery to the Lungs Conference. CF mucus affects the deposition pattern of aerosols on the epithelial surface of the upper respiratory tract and poses a barrier to the effective diffusion of drugs in the CF lung. To improve drug delivery in CF it is vital to reduce or remove this barrier. The inventors have unexpectedly found that the composition of the disclosure causes the dispersal of actin in the mucus resulting in the dispersal of mucus. This can help open up the airway passages facilitating the delivery of the drugs in the lungs.

Accordingly, in one aspect, there is provided a method to disperse actin in the mucus of a subject, comprising, or alternatively consisting essentially of, or yet further consisting of, administering to the subject an effective amount of the composition disclosed herein, thereby dispersing the actin in the mucus of the subject. In another aspect, there is provided a method to treat a disease caused by an aggregation of actin in the mucus of a subject, comprising, or alternatively consisting essentially of, or yet further consisting of, administering to the subject a therapeutically effective amount of a composition disclosed herein, thereby treating the disease in the subject. In some embodiments, the disease is cystic fibrosis.

Asthma is a chronic obstructive lung disorder where activation of the immune system by antigens leads to allergic inflammation. When this type of immune activation occurs it is accompanied by pulmonary inflammation, bronchial hyper responsiveness, goblet cell and submucosal gland hyperplasia, and mucin over-production and hyper-secretion. Mucus over-production and plugging associated with goblet cell and submucosal gland cell hyperplasia is a part of the pathology of asthma.

Accordingly, in one aspect, there is provided a method to treat asthma in a subject by dispersing an aggregation of mucin in the subject, comprising, or alternatively consisting essentially of, or yet further consisting of, administering to the subject a therapeutically effective amount of a composition disclosed herein, thereby treating the asthma in the subject.

Chronic bronchitis is another form of chronic obstructive pulmonary disorder which is defined as the chronic over-production of sputum. Mucus over-production is generally associated with inflammation of the conducting airways. The increased production of mucus is associated with airway obstruction, which is one of the cardinal features of this pulmonary disorder. Decongestants, expectorants and combinations of these agents that are often used to treat the symptoms of bronchitis are not effective in altering the mucin production.

Accordingly, in one aspect, there is provided a method to treat chronic bronchitis in a subject by dispersing an aggregation of mucin in the subject, comprising, or alternatively consisting essentially of, or yet further consisting of, administering to the subject a therapeutically effective amount of a composition disclosed herein, thereby treating the chronic bronchitis in the subject.

Chronic obstructive pulmonary disease (COPD) is a progressive disease that makes it hard for the subject to breathe. “Progressive” means the disease gets worse over time. COPD can cause coughing that produces large amounts of mucus, wheezing, shortness of breath, chest tightness, and other symptoms. Cigarette smoking is the leading cause of COPD. Long-term exposure to other lung irritants, such as air pollution, chemical fumes, or dust, also may contribute to COPD.

In yet another aspect, there is provided a method to treat COPD in a subject by dispersing an aggregation of mucin in the subject, comprising, or alternatively consisting essentially of, or yet further consisting of, administering to the subject a therapeutically effective amount of a composition disclosed herein, thereby treating the COPD in the subject.

Inflammation of the inner ear (OM) is common in children as well as in adults. Recurrent, protracted ear inflammation causes severe pain, restlessness and muffled hearing, which can hamper development and prejudice learning. Blockage of the Eustachian tube (OT) which links the middle ear cavity to the oral cavity can be a main cause of inflammation or a secondary factor—caused by inflammation and swelling of the mucus membrane of the ear and blocking the Eustachian canal. Opening the Eustachian tube is an important and essential step towards healing the inflammation. Blockage of the canal prevents drainage of the fluids secreted by the mucosal membrane of the middle ear and prevents equalization of the atmospheric pressure on either side of the eardrum. This increases the pressure inside the ear cavity and pushes the eardrum outwards, leading to pain, muffled hearing, and occasionally problems with balance. Moreover, increased pressure in the middle ear cavity prevents the absorption of medication into the region via local osmosis (eardrops) or systemically (via the blood).

Accordingly, in one aspect, there is provided a method to treat inflammation of the inner ear in a subject by dispersing an aggregation of mucin in the subject, comprising, or alternatively consisting essentially of, or yet further consisting of, administering to the subject a therapeutically effective amount of a composition disclosed herein, thereby treating the inflammation of the inner ear in the subject.

Over-production of mucin is also implicated in the disorders of the eye, such as, but not limited to, conjunctivitis. The ocular mucins are relatively large molecules, and have a significant role in the gel-forming nature of the tear film. The viscoelasticity of the tear film derives from the specific structure and gel-forming properties of the ocular mucins, and allows the tear film to absorb the shear force of the blink, which would otherwise irritate and damage the ocular surface. The trans membrane mucin, on the other hand, serves more as a protective layer on the actual cellular surface of the ocular epithelium, functioning to directly protect and lubricate the ocular surface, as well as to anchor the highly hydrated gel (mucus) of the tear film gel-forming mucins, thereby assisting in the spreading and stability of the tear film over the ocular surface. An excess of mucin is therefore expected to affect the lubricating, protective, barrier and other functions of the mucins at the mucosal surface.

Conjunctivitis is an inflammation of the eye common in children and adults. Conjunctivitis can be caused by a virus, bacteria, irritating substances (shampoos, dirt, smoke, and especially pool chlorine), allergens (substances that cause allergies) or sexually transmitted diseases (STDs). Conjunctivitis results in redness of the eye and a thick yellow discharge that crusts over the eyelashes, especially after sleep. The washing of the eye with the composition of the disclosure can reduce the crusting of the mucus inside the eye, thereby providing relief. The composition is especially useful for pediatric use since it is convenient to use and is free of any chemicals. Accordingly, in one aspect, there is provided a method to treat inflammation of the eye in a subject by dispersing an aggregation of mucin in the subject, comprising, or alternatively consisting essentially of, or yet further consisting of, administering to the subject a therapeutically effective amount of a composition disclosed herein, thereby treating the inflammation of the eye in the subject.

In some embodiments, the dispersion of the aggregate of the mucin in the methods provided herein, results in a reduction of a size of the mucin. In some embodiments, the dispersion of the aggregate of the mucin results in a reduction of a size of the mucin to less than about 10 μm in from about 5 hour to about 1 hour. In some embodiments, the dispersion of the aggregate of the mucin results in a reduction of a size of the mucin to less than about 10 μm in about 1-5 hour; less than about 10 μm in about 1 hour; less than about 9 μm in about 1-5 hour; less than about 9 μm in about 1 hour; less than about 8 μm in about 1-5 hour; less than about 8 μm in about 1 hour; less than about 7 μm in about 1-5 hour; less than about 7 μm in about 1 hour; less than about 6 μm in about 1-5 hour; less than about 6 μm in about 1 hour; less than about 5 μm in about 1-5 hour; less than about 5 μm in about 1 hour; less than about 4 μm in about 1-5 hour; less than about 4 μm in about 1 hour; less than about 3 μm in about 1-5 hour; less than about 3 μm in about 1 hour; less than about 2 μm in about 1-5 hour; less than about 2 μm in about 1 hour; less than about 1 μm in about 1-5 hour; or less than about 1 μm in about 1 hour.

### Formulations and Dosages

In one aspect, the disclosure provides compositions for use in the methods described herein. In some embodiments, the compositions comprise, or alternatively consist essentially of, or yet further consist of nanoparticles wherein the average diameter of the nanoparticles is less than about 500 nm. In one aspect the plurality have an overall negative charge. In another aspect, the plurality of nanoparticles have an overall positive charge. In some embodiments, the composition further comprises, or alternatively consists essentially of, or yet further consists of a pharmaceutically acceptable carrier. In another aspect, the compositions contain carriers that modulate (controlled release) the release of the nanoparticle when administered to a subject in need thereof. In a further aspect, the compositions are suitable for topical application to the mucosal surface of a subject in need of such treatment.

The pharmaceutical compositions of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations. The pharmaceutical composition of the disclosure is also contemplated to be administered as a suppository.

The compositions of this disclosure are formulated for pharmaceutical administration to a mammal, preferably a human being. Such pharmaceutical compositions of the disclosure may be administered in a variety of ways, preferably topically.

In some embodiments, therapeutic composition containing the nanoparticles of this disclosure is administered by contacting the composition with the mucous or mucous-producing cells in the respiratory airways. In some embodiments, the composition of the disclosure is administered to the subject by inhalation such as nasal inhalation or pulmonary inhalation. The administration of therapeutic compositions to the respiratory airways is a well developed art in the field, and such methods are applicable here. In some embodiments, an aerosolized or nebulized (vaporous) liquid composition containing an effective amount of the nanoparticles of the disclosure, is delivered to the respiratory airways by breathing in the vaporous composition, or by forced (pressurized) periodic inflation breathing of the lungs with the vapor. In some embodiments, an expectorant composition containing the nanoparticles of the disclosure, is delivered to the throat or the gastro intestinal tract of the subject.

The compositions are administered in a manner compatible with the dosage formulation, and in a therapeutically effective amount. The quantity to be administered and treatment regimen depends on the subject to be treated such as the age, body weight, general health, sex and diet, renal and hepatic function of the subject, capacity of the subject's system to utilize the active ingredient, the particular indication being treated, the mode of administration, the time of administration, rate of excretion, drug combination, the severity of the indication being treated, and degree of therapeutic effect desired. Precise amounts of active ingredient required to be administered depend on the judgment of the practitioner and are peculiar to each individual. Determination of an effective dosage is well within the capabilities of those skilled in the art. However, suitable dosage ranges for systemic application are disclosed herein and depend on the manner of administration. Suitable regimes for administration are also variable, but are typified by an initial administration followed by repeated doses at one or more hour intervals. Alternatively, continuous delivery of an aerosolized or nebulized composition during continuous breathing sufficient to bathe the respiratory airways are contemplated.

Thus, the administration of the composition can be in the form of a single unit dose of inhalation or expectorant, multiple inhalations or doses of expectorant, or during continuous breathing of the vapors. Alternatively, a lavage of the lungs may be utilized whereby the lavage solution contains the composition of the disclosure. The term “unit dose” when used in reference to a therapeutic composition of the present disclosure refers to physically discrete units suitable as unitary dosage for the subject, each unit containing a predetermined quantity of active material calculated to produce the desired therapeutic effect in association with the required diluent, i.e., carrier, or vehicle.

In one aspect, there is provided a device for a delivery of a composition for dispersing mucin, wherein the device comprises an effective amount of a composition comprising a nanoparticle of the disclosure. In some embodiments, means for delivering a therapeutic composition comprises a device which produces an aerosol of a liquid composition. Such devices are generally well known in the art. These devices can be nebulizers, small particle aerosol generators, metered-dose inhalers, inhalers with a propellant, and the like devices. The devices may be adapted to deliver the therapeutic compositions of the disclosure in the form of a finely dispersed mist of liquid, foam or powder. The devices may use any propellant system known to those in the art including, but not limited to, pumps, liquefied-gas, compressed gas and the like. Devices of the present disclosure typically comprise a container with one or more valves throw which the flow of the therapeutic composition travels and an actuator for controlling the flow. Suitable devices for use in the present disclosure may be seen in, for example, in Remington: The Science and Practice of Pharmacy, 19th Edition, Chapter 95, pages 1676-1692, Mack Publishing Co., Easton, Pa. 1995.

An exemplary nebulizer includes an Acorn II jet nebulizer, a Marquest or DeVilbiss 646 nebulizer, a compressed air generator such as a Pulmaide, a Devilbiss, or an IPPB device, which typically nebulize 3-10 milliliters (mL) of solution over about 5-20 minutes, and the like commercially available nebulizers. Aerosol droplets produced by nebulizers are typically of a size that deposits the aerosolized droplets in the larger bronchioles of the lung. Alternatively, a small particle aerosol generator, such as the commercially available SPAG-2 or Viratek, generates smaller droplets which are deposited more distally in the airways, such as in the ducts and sacs.

The duration and frequency of the administration of a therapeutic composition can vary widely depending upon the severity of the symptoms and infectious state. Typical dosages can be from one unit dose up to a continuous contacting dose over a period of from one to several days. Thus, the contacting can follow a variety of regimens. Exemplary regimens for inhalation include one or more unit dose, administrations over time to continuously inhaled aerosols for prolonged periods of from 5 minutes up to several hours or even days. More frequent administrations may be used under conditions of rapid cell turnover, such as during infection.

Effective dosages can be estimated initially from in vitro assays. For example, an initial dosage for use in animals can be formulated to achieve a local (topical) or circulating blood or serum concentration of nanoparticles that is at or above an IC₅₀ of the particular nanoparticle as measured in as in vitro assay. Calculating dosages to achieve such circulating blood or serum concentrations taking into account the bioavailability of the particular nanoparticles is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, and the references cited therein.

Initial dosages can also be estimated from in vivo data, such as animal models. Animal models useful for testing the efficacy of nanoparticles to treat or prevent the various diseases described above are known in the art. Suitable animal models include, but are not limited to mouse, pigs, monkeys, and frogs. Ordinarily skilled artisans can routinely adapt such information to determine dosages suitable for human administration.

The amount of nanoparticle that can be administered to the subject will typically be in the range of from about 200 μg/m³ to about 1 μg/m³; or about 150 μg/m³ to about 1 μg/m³; or about 100 μg/m³ to about 1 μg/m³; or about 50 μg/m³ to about 1 μg/m³; or about 10 μg/m³ to about 1 μg/m³. In some embodiments, the dosage of nanoparticles in the solution is in the range from about 100 mg/L to about 10 g/L.

The amount can be higher or lower, depending upon, among other factors, the activity of the nanoparticle, its bioavailability, the mode of administration, and various factors discussed above. Dosage amount and interval can be adjusted individually to provide plasma levels of the nanoparticles which are sufficient to maintain therapeutic or prophylactic effect. For example, the nanoparticles can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of nanoparticles may not be related to plasma concentration. Skilled artisans will be able to optimize effective local dosages without undue experimentation.

Preferably, the agents and/or compositions will provide therapeutic or prophylactic benefit without causing substantial toxicity. Toxicity of the nanoparticles can be determined using standard pharmaceutical procedures. The dose ratio between toxic and therapeutic (or prophylactic) effect is the therapeutic index. The nanoparticles that exhibit high therapeutic indices are preferred. The compositions of the disclosure are of sufficiently high pharmaceutical quality, i.e., they are pharmaceutically stable over a storage time of some years, preferably at least one year, more preferably two years.

The pH of the composition may be adjusted by the addition of pharmacologically acceptable acids. Pharmacologically acceptable inorganic acids or organic acids may be used for this purpose. Examples of preferred inorganic acids are selected from the group consisting of hydrochloric acid, hydrobromic acid, nitric acid, sulphuric acid and phosphoric acid. Examples of particularly suitable organic acids are selected from the group consisting of ascorbic acid, citric acid, malic acid, tartaric acid, maleic acid, succinic acid, fumaric acid, acetic acid, formic acid and propionic acid. Preferred inorganic acids are hydrochloric acid and sulphuric acid. If desired, mixtures of the abovementioned acids may also be used, particularly in the case of acids which have other properties in addition to their acidifying properties, e.g. those which act as flavorings or antioxidants, such as for example citric acid or ascorbic acid. If desired, pharmacologically acceptable bases may be used to titrate the pH precisely. Suitable bases include for example alkali metal hydroxides and alkali metal carbonates. If bases of this kind are used, care must be taken to ensure that the resulting salts, which are then contained in the finished pharmaceutical formulation, are pharmacologically compatible with the abovementioned acid.

Other pharmacologically acceptable excipients may also be added to the composition according to the disclosure. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilisers, antioxidants and/or preservatives which prolong the shelf life of the finished pharmaceutical formulation, as well as flavorings, vitamins and/or other additives known in the art. The additives also include pharmacologically acceptable salts such as sodium chloride, for example. The preferred excipients include antioxidants such as ascorbic acid, vitamin A, vitamin E, tocopherols and similar vitamins or provitamins occurring in the human body.

Preservatives can be added to protect the formulation from contamination with pathogenic bacteria. Suitable preservatives are those known from the prior art, particularly benzalkonium chloride or benzoic acid or benzoates such as sodium benzoate in the concentration known from the prior art. The amount of benzalkonium chloride is between 1 mg and 50 mg per 100 ml of formulation, about 7 to 15 mg per 100 ml, or about 9 to 12 mg per 100 ml of the formulation according to the disclosure.

In some embodiments, it may be important to ensure homogenous dispersion of the nanoparticles in suspension, without the formation of aggregates for correct aerosolization. The formation of more or less compact aggregates can give rise to problems of distribution and therefore of uniformity of dose during the filling of the containers.

In some embodiments, the composition according to the disclosure is sterile. In some embodiments, the composition of the disclosure is devoid of preservatives and bacteriostatics. This can prevent the induction of the allergic reactions or irritation of the respiratory mucosa in the subjects. Various processes can be used to manufacture sterile pharmaceutical formulations. For example, the active ingredient can be sterilized by dry heating or irradiation such as with gamma rays, followed by preparation of the formulation under aseptic conditions, or the formulation can be pre-prepared and sterilized by treatment in an autoclave or by filtration.

The agents and compositions of the present disclosure can be used in the manufacture of medicaments and for the treatment of humans and other animals by administration in accordance with conventional procedures, such as an active ingredient in pharmaceutical compositions.

### Drugs Screens and Assays

In one aspect, there is provided an in vitro method to disperse mucin or actin, comprising, or alternatively consisting essentially of, or yet further consisting of administering to mucin or actin a composition comprising a nanoparticle. In some embodiments, the size of the nanoparticle is less than about 500 nm. In some embodiments, the nanoparticle is negatively charged, while in other aspects the nanoparticle is positively charged. When nanoparticles are being introduced into cells suspended in a cell culture, the cells are incubated together with the nanoparticle in an appropriate growth media, for example Luria broth (LB) or a suitable cell culture medium. When in vitro experiments are to be performed, nanoparticles can be added directly to a selected cell growth medium before cells are introduced into the medium. Such a medium may be compatible not only with the physiological requirements of the cells, but also with the chemical and reactivity profile of the nanoparticles. The nanoparticles's profile will be apparent to one of skill in the art upon review of the present disclosure.

In some embodiments, there is provided an in vitro method to screen for nanoparticles that disperse mucin, comprising, or alternatively consisting essentially of, or yet further consisting of (i) administering a composition comprising a candidate nanoparticles to a test sample containing mucin; (ii) determining the dispersal of mucin in the test sample; (iii) comparing the dispersal of mucin in the test sample with a control cell where the control cell does not have the nanoparticles in the composition, or the control cell has nanoparticles that disperse mucin, thereby screening for the candidate nanoparticles that disperse mucin. The methods to determine the dispersal of mucin in a sample include, but are not limited to, dynamic laser scattering that determines the size of the mucin before and after dispersal.

In some embodiments, there is provided an in vitro method to screen for nanoparticles that disperse actin, comprising, or alternatively consisting essentially of, or yet further consisting of (i) administering a composition comprising a candidate nanoparticles to a test sample containing actin; (ii) determining the dispersal of actin in the test sample; (iii) comparing the dispersal of actin in the test sample with a control cell where the control cell does not have the nanoparticles in the composition, or the control cell has nanoparticles that disperse actin, thereby screening for the candidate nanoparticles that disperse actin. The methods to determine the dispersal of actin in a sample include, but are not limited to, dynamic laser scattering that determines the size of the actin before and after dispersal.

In some embodiments of the in vitro methods, a composition comprising a calcium ion or any other suitable positively charged ion may be added to mucin to cause the aggregation of mucin in the sample. The composition of the nanoparticle, as provided herein, is then added to the aggregated mucin to disperse the mucin.

For the in vitro methods, mucins or actin can be derived from a wide variety of “natural” sources including, but not limited to porcines, bovines, goats, sheep, cattle, felines, non-human primates, humans, and the like. Alternatively, mucins can be chemically prepared. Mucins are selected from buccal and gastrointestinal mucins. The term buccal and gastrointestinal mucins are intended to designate any mucin which is present in the oral cavity or in the gastrointestinal system, respectively. Typical examples are mucins from salivary glands and gastric mucins.

The mucin concentration is selected to reflect the mucin concentration of, and hence to achieve a viscosity similar to, the naturally occurring oral fluid(s) for which the oral fluid standard stands as a surrogate. Thus, in pathological conditions where oral fluid viscosity is abnormal, the mucin concentration of the oral fluid standard will be adjusted to approximate the viscosity of the abnormal oral fluid to provide a standard for testing methods and devices used in the pathological patient. Conversely, where oral fluid viscosity is “normal”, mucin concentrations of the oral fluid standard will be adjusted to reflect normal oral fluid viscosity.

Mucins or actins can also be obtained from commercial sources (e.g. Sigma Chemical Co., St. Louis, Mo., USA)) or, as indicated above, isolated directly from various non-human mammals. Methods of isolating mucins or actins are well known to those of skill in the art. For example, porcine gastric mucin is typically obtained as a by-product in the production of pepsin from hog stomachs. The mucin can be additionally purified by multiple alcohol precipitations, such as 2-3 precipitations with 60% ethanol. During the precipitations, and during the manipulation of the mucin, the use of gentle conditions will result in minimizing of viscosity-decreasing degradation.

In the in vitro methods of the disclosure, the delivery of the nanoparticles to the mucin can be detected on both the interior and exterior of cells or mucin in a variety of ways. One method of detecting the presence of a nanoparticles is by monitoring a sample for the homeostatic change the nanoparticle delivery is designed to produce. For some applications, however, it might be desirable to monitor the presence of a nanoparticle delivery by methods including, but are not limited to, the use of transmission electron, fluorescence and other microscopy techniques; spectroscopic-based detection; and detection methods involving proteins, such as immunological methods.

Transmission electron microscopy (TEM) can be used to determine the presence of a nanoparticles. Nanoparticles of size about 5 nm and larger can be clearly visualized by TEM. TEM is a useful method of detect the presence and subcellular localization of nanoparticles. TEM can also be used to estimate the density of nanoparticles in a region. A density calculation can be performed by counting the number of observed particles in a given area scanned by TEM. An understanding of the density of nanoparticles in a defined region, such as a cell's nucleus or cytoplasm, can provide information regarding the size requirements for a nanoparticle, the effectiveness of a given nuclear localization signal and other parameters.

Nanoparticles of the disclosure can also be detected spectroscopically. Ultraviolet (UV), visible and infrared (IR) spectroscopic methods can be employed. The choice of detection method will typically depend on the experimental design. In one embodiment, nanoparticles of the disclosure can be indirectly detected using fluorescence spectroscopy. Expression of GFP and other fluorescent marker proteins provided by the nanoparticles can be detected by fluorescence and can act as an indicator of the presence of a nanoparticles. A fluorescent moiety can be associated with the nanoparticle and the presence of the nanoparticle itself can be identified.

Microscopy techniques such as bright field microscopy, phase contrast microscopy, confocal microscopy and other techniques can be employed to detect the presence of nanoparticles. Phase contrast microscopy is typically used for the visualization of cellular organelles, and can be employed to detect the presence of nanoparticles. Confocal microscopy can also be useful for detecting nanoparticles. The resolution of any of the above microscopy techniques can be enhanced by the introduction of various contrast enhancement or other agents known to refine images and increase resolution.

Protein-based detection of a nanoparticle is also possible. For example, a labeled protein is bound to the nanoparticle. Alternatively, a first protein is attached to the nanoparticle. Then a second protein known to associate with a first protein bound to a nanoparticle can be labeled and used as a probe. Suitable labels include fluorescent moieties and other labels. Upon association of the first and second proteins, and therefore association of the labeled second protein on the nanoparticle, the presence of the nanoparticle is detectable by detecting the presence of the probe. Any suitable protein pair can be used to detect a nanoparticle of the disclosure.

### Kits

In yet another aspect, this disclosure provides a kit comprising, or alternatively consisting essentially of, or yet further consisting of: a composition comprising a nanoparticle wherein the average diameter of the plurality of nanoparticles is less than about 500 nm; and instructions for use. In some embodiments, the composition comprises an effective amount of the nanoparticle in a pharmaceutically acceptable carrier. In some embodiments, the pharmaceutically acceptable carrier in the kits is suitable for topical administration of the agent. The formulations can be for immediate or controlled release of the active ingredients. In some embodiments, the pharmaceutically acceptable carrier further comprises, or alternatively consists essentially of, or yet further consist of, a penetration or permeation enhancer.

Also provided are kits for administration of the compounds for treatment of disorders as described herein. Kits may further comprise suitable packaging and/or instructions for use of the compound. Kits may also comprise a means for the delivery of the composition and instructions for administration. Alternatively, the kit provides the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, a metered dose inhaler or a nebulizer.

The kits may include other therapeutic compounds for use in conjunction with the compounds described herein. The other therapeutic compounds include, but are not limited to, conventional pain-killers, antihistamines, etc. These compounds can be provided in a separate form or mixed with the compounds of the present disclosure.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the disclosure, kits for treating a subject who suffers from or is susceptible to the conditions described herein are provided, comprising a container comprising a dosage amount of a composition, as disclosed herein, and instructions for use. The container can be any of those known in the art and appropriate for storage and delivery of oral, intravenous, topical, or inhaled formulations. Kits may also be provided that contain sufficient dosages of the effective composition or compound to provide effective treatment for an individual for an extended period, such as a week, 2 weeks, 3, weeks, 4 weeks, 6 weeks, or 8 weeks or more.

The following examples are provided to illustrate select embodiments of the disclosure as disclosed and claimed herein.

### EXAMPLES

### Example 1

### Effect of Nanoparticles on Mucin

Negatively charged polystyrene nanoparticles (120 nm) were used to disperse mucin gels. Mucin was first gelated to approximately 8 μm by crosslinking with 8 mM of Ca²⁺ in Hanks' solution for 48 hours (hrs). Dynamic laser scattering was used to measure the size of mucin aggregates after crosslinking with Ca²⁺. Negatively charged polystyrene nanoparticles (120 nm) at 10 mg/L were subsequently added to disperse the mucin gels. The size of mucin gels reduced to 1 μm (approximately 7 folds) in under 1 hr. The reduction in size was continuously monitored (at 1 hr, 3 hr, 5 hr and 24 hr) and remained at 1 μm after 24 hrs.

### Example 2

### Effect of Nanoparticles on Actin

### Materials:

The G-actin (10 mg/ml) and Alex488 labeled G-actin (8.6 mg/ml) monomers were purchased from Cytoskeleton, Inc. (Denver, Colo.). The F-buffer contained 4.5 mM Tri-HCl, 0.18 mM CaCl₂, 50 mM KCl, 2 mM MgCl₂, 1.2 mM ATP and 0.4 5 mM DTT. The G-buffer contained 5 mM Tris-HCl and 0.2 mM CaCl₂. Polystyrene Nanoparticles with three different types of surface modifications were purchased from Bangs Laboratories, Inc. The 23 nm polystyrene nanoparticles, 57 nm polystyrene nanoparticles with amine surface modification, and 24 nm polystyrene nanoparticles with carboxyl surface modification were used.

### Methods:

To investigate the influence of nanoparticles on F-actin network, the F-actin filaments and MgCl₂ were used to form the F-actin Network. Both G-actin (10 mg/ml) and Alex488 labeled G-actin (8.6 mg/ml) were diluted with G-buffer into 2 mg/ml concentration. After mixing the G-actin and Alex488 labeled G-actin, F-buffer was added to polymerize G-actin into F-actin filament. The concentration of F-actin filament was 0.5 mg/ml. After gentle mixing 1M MgCl₂ solution with F-actin filaments solution, the formation of F-actin network under fluorescent microscope was observed. The nanoparticle solutions was injected on the F-actin network and the morphology change of the network was observed. In the experiments, the effects of two different nanoparticle concentrations, 10 g/L and 100 mg/L, is shown. The data are listed below.

### Experiment Data:

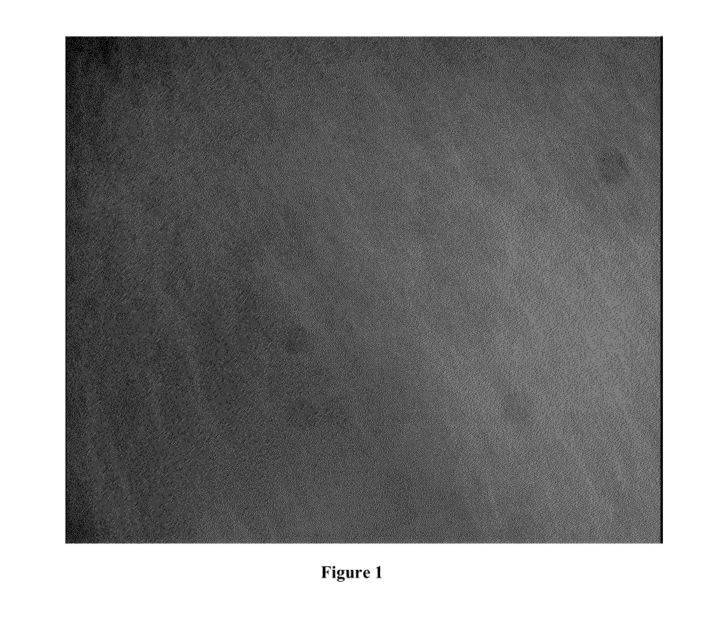

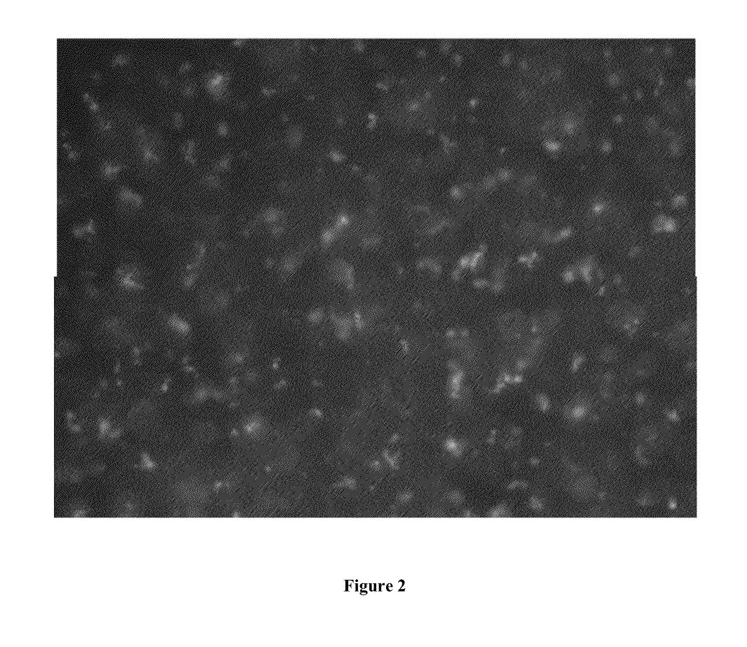

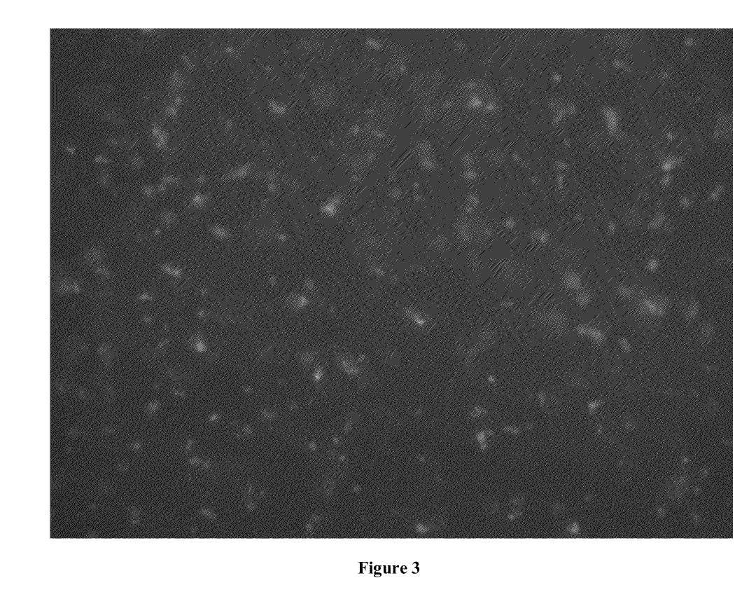

Control Group: FIG. 1 illustrates F-actin network (F-actin+MgCl₂), before adding any nanoparticles. FIGS. 2-7 illustrate an effect of nanoparticles on F-actin network. FIGS. 2 and 3 illustrate the effect of hydrophobic polystyrene nanoparticles on F-actin network where FIG. 2 is for higher concentration of polystyrene nanoparticles (10 g/L) and FIG. 3 is for lower concentration of polystyrene nanoparticles (100 mg/L). FIGS. 2 and 3 show that hydrophobic nanoparticles disperse actin filaments.

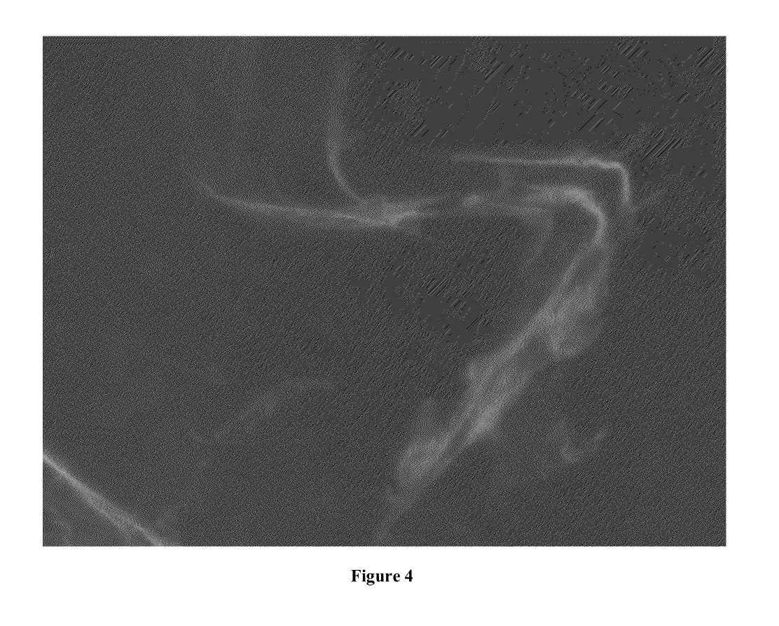

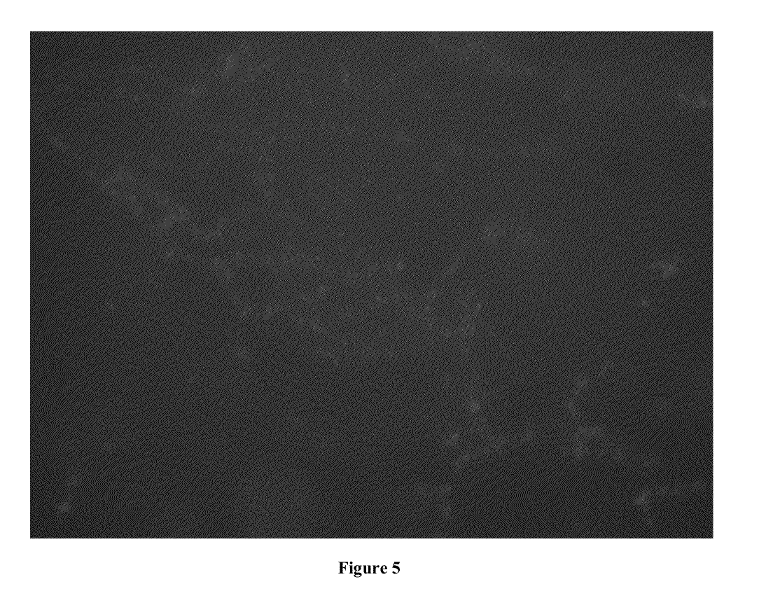

FIGS. 4 and 5 illustrate the effect of positively charged amine surface modified polystyrene nanoparticles on F-actin network where FIG. 4 is for higher concentration of polystyrene nanoparticles (10 g/L) and FIG. 5 is for lower concentration of polystyrene nanoparticles (100 mg/L). FIGS. 4 and 5 show that positively charged nanoparticles cross-link actin filaments at high concentration, but disrupt actin filaments at low concentration.

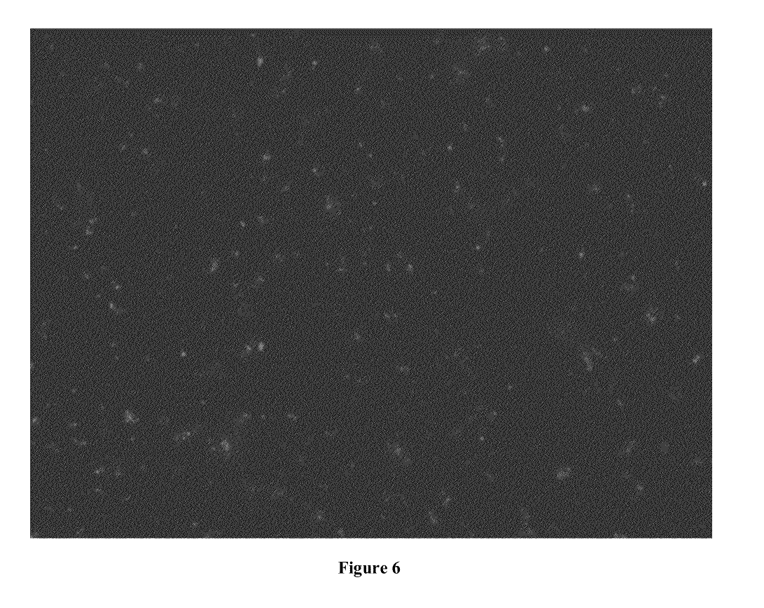

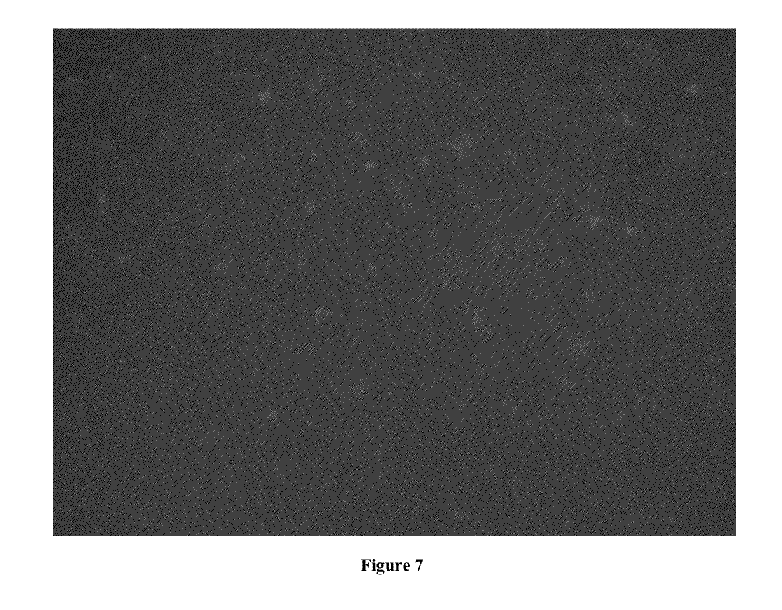

FIGS. 6 and 7 illustrate the effect of negatively charged carboxyl surface modified polystyrene nanoparticles on F-actin network where FIG. 6 is for higher concentration of polystyrene nanoparticles (10 g/L) and FIG. 7 is for lower concentration of polystyrene nanoparticles (100 mg/L). FIGS. 6 and 7 show that negatively charged nanoparticles disperse actin filaments at both high and low concentration. The dispersion induced by negative nanoparticles is more profound than hydrophobic nanoparticles.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method to disperse mucin, actin, or both, said method comprising contacting the mucin, actin, or both with an effective amount of negatively charged nanoparticles, thereby dispersing the mucin, actin, or both.

2. The method of claim 1, wherein the contacting and dispersal is of mucin.

3. The method of claim 1, wherein the contacting is in vitro.

4. The method of claim 1, wherein the contacting is in vivo.

5. The method of claim 3, wherein the contacting is by topical, inhalation, buccal, rectal, vaginal, nasal, oral, ocular, or parenteral administration of said nanoparticles.

6. The method of claim 5, wherein said administration is by inhalation of said nanoparticles.

7. The method of claim 6, wherein the subject is suffering from a respiratory disease selected from the group consisting of influenza, viral infection, common cold, inflammation of the trachea or lungs, asthma, cystic fibrosis, chronic bronchitis, pneumonia, and chronic obstructive pulmonary disease.

8. The method of claim 5, wherein the subject is suffering from a condition selected from the group consisting of inflammation of an eye, inflammation of the middle ear, mucosal clogging of the rectum, and mucosal clogging of the vaginal tract.

9. The method of claim 1, wherein said nanoparticles are polystyrene.

10. The method of claim 1, wherein said nanoparticles have an average diameter of less than about 500 nm.

11. The method of claim 1, wherein said dispersing causes reduction of size of an aggregate of said mucin, actin, or both.

12. The method of claim 11, wherein said dispersing reduction of size of an aggregate of mucin.

13. The method of claim 1, wherein said nanoparticles are not attached to a drug.

14. The method of claim 1, wherein said nanoparticles are biologically inert.

15. The method of claim 1, wherein said nanoparticles have negatively charged surface carboxyl groups.

16. A method of relieving symptoms caused by aggregation of mucin in a subject in need thereof, said method comprising administering to said subject an effective amount of negatively charged nanoparticles in a manner allowing said negatively charged nanoparticles to contact said aggregated mucin, thereby dispersing said aggregated mucin in said subject.

17. The method of claim 16, wherein said administration is topical, buccal, rectal, vaginal, nasal, oral, ocular, parenteral, or by inhalation.

18. The method of claim 17, wherein said administration is by inhalation.

19. The method of claim 18, wherein the subject is suffering from a respiratory disease selected from the group consisting of influenza, viral infection, common cold, inflammation of the trachea or lungs, asthma, cystic fibrosis, chronic bronchitis, pneumonia, and chronic obstructive pulmonary disease.

20. The method of claim 19, wherein the respiratory disease is cystic fibrosis.

21. The method of claim 17, wherein the subject is suffering from a condition selected from the group consisting of inflammation of an eye, inflammation of the middle ear, mucosal clogging of the rectum, and mucosal clogging of the vaginal tract.

22. The method of claim 16, wherein said nanoparticles are made of polystyrene.

23. The method of claim 16, wherein said nanoparticles have an average diameter of less than about 500 nm.

24. The method of claim 16, wherein said nanoparticles are not attached to a drug.

25. The method of claim 16, wherein said nanoparticles are biologically inert.

26. The method of claim 16, wherein said nanoparticles have negatively charged surface carboxyl groups.
